(19) United States
(12) Patent Application Publication
(10) Pub. No.: US 2024/0168018 A1
(43) Pub. Date: May 23, 2024

(54) METHODS FOR DETECTING AND EVALUATING VIRUSES AND VIRUS-LIKE PARTICLES

(71) Applicant: Regeneron Pharmaceuticals, Inc., Tarrytown, NY (US)

(72) Inventors: Michael Rosconi, New City, NY (US); Nina Liu, Chappaqua, NY (US); Erica Pyles, New City, NY (US)

(21) Appl. No.: 18/389,375

(22) Filed: Nov. 14, 2023

Related U.S. Application Data
(60) Provisional application No. 63/426,554, filed on Nov. 18, 2022.

(51) Int. Cl. G01N 33/569; G01N 21/64; G01N 33/58

(52) U.S. Cl. CPC G01N 33/56983; G01N 21/6428; G01N 33/582; G01N 2021/6439; G01N 2333/015

(57) ABSTRACT

The present inventions provide methods for detecting and evaluating viruses and virus-like particles using A4F combined with Multi-Angle Light Scattering (MALS) and fluorescent (Flr) detectors. Systems for performing the methods also are provided.

This application claims priority to U.S. Ser. No. 63/426,554, filed Nov. 18, 2022, which is hereby incorporated by reference.

### FIELD OF THE INVENTIONS

The present inventions provide methods for detecting and evaluating viruses and virus-like particles using A4F combined with fluorescent (Flr) detectors and optionally Multi-Angle Light Scattering (MALS).

### BACKGROUND OF THE INVENTIONS

Biological therapy is increasingly being used world-wide to treat medical disorders. Adeno-associated virus (AAV) is allowing for genetic therapy to treat gene-based disorders by using AAVs modified with transgenes, which is a gene other than AAV Rep and Cap genes. A typical type of transgene would be a mammalian gene, such as a human gene.

Virus-like particles (VLPs) comprising chimeric surface proteins, such as viral spike proteins from non-native origins, can be used to study biological therapeutics. In most instances, the surface proteins come from a source that is different from the virus that the VLP is based upon. The development of biological therapies has created a need for advanced analytical capabilities to facilitate design, production, quality control of biological products, as well as the ability to evaluate these therapies in biological backgrounds.

Asymmetric flow field flow-fractionation (A4F, also referred to as AF4) paired with a detector, such as ultraviolet light (UV) or Multi-Angle Light Scattering (MALS) is a powerful tool for separating and analyzing large molecules and large macromolecular complexes. These detectors, however, are susceptible to interference from other proteinaceous components that are typically present in certain fluids, such as biological fluids.

Accordingly, there are needs to detect and evaluate viruses and virus-like particles in various fluids. These needs were unmet until the present inventions.

### SUMMARY OF THE INVENTIONS

The inventions provide methods for characterizing virus-like particles and viral glycoproteins using asymmetric flow field flow-fractionation (A4F) and at least two types of detectors, wherein the methods comprise the steps of: (A) fractionating by A4F a first virus-like particle sample; and determining at least one of molar mass and size distribution of the virus-like particle and viral glycoproteins in the first virus-like particle sample using Multi-Angle Light Scattering (MALS); and (B) fractionating by A4F a second virus-like particle sample further comprising a fluorescence labeled detection reagent; and detecting in the second virus-like particle sample the free glycoproteins and virus-like particle associated viral glycoproteins using a fluorescence detector, wherein (A) and (B) can be performed consecutively in any order or simultaneously, and (C) comparing the elution time profiles from (A) and (B). The viral glycoproteins can be spike proteins, such as spike proteins from influenza, coronavirus and filamentous virus, for example Ebola. The virus-like particles can be based on a Parvoviridae (such as adeno-associated virus), Retroviridae (such as lentivirus), Flaviviridae (such as Hepatitis C virus), Paramyxoviridae (such as Nipah), Adenoviridae (such as adenovirus), vesicular stromatitis virus (VSV) and bacteriophages (such as Qβ), for example. The samples can be from the same preparation or different preparations.

The inventions further provide methods for characterizing retargeted adeno-associated virions (retargeted AAV) and retargeting molecules using asymmetric flow field-flow fractionation (A4F) and at least two types of detectors, wherein the methods comprise the steps of: (A) fractionating by A4F a first retargeted AAV sample; and determining at least one of molar mass and size distribution of retargeted adeno-associated virions and retargeting molecules using Multi-Angle Light Scattering (MALS); and (B) fractionating by A4F a second retargeted AAV sample further comprising a fluorescence labeled detection reagent; and detecting the retargeted adeno-associated virions and retargeting molecules in the second retargeted AAV sample using a fluorescence detector, wherein (A) and (B) can be performed consecutively in any order or simultaneously, and (C) comparing the elution time profiles from (A) and (B). The retargeting molecule can be an antibody, such as a bispecific antibody. The AAV can be recombinant and comprise a transgene. The AAV can be a recombinant AAV based upon AAV1, 2, 3, 4, 5, 6, 7, 8, 9, 10, 11, 12, 13, rh74, and others, including any variants thereof. The samples can be from the same preparation or different preparations.

The inventions further provide methods for characterizing (i) particles selected from the group consisting of viruses and virus-like particles and (ii) anti-particle Fc-containing proteins, wherein the characterizing is by using asymmetric flow field-flow fractionation (A4F) and at least two types of detectors, wherein the methods comprise the steps of: (A) fractionating by A4F a first particle sample; and determining at least one of molar mass and size distribution of particles and anti-particle Fc-containing proteins using Multi-Angle Light Scattering (MALS); and (B) fractionating by A4F a second particle sample further comprising a fluorescence labeled detection reagent; and detecting the particles and anti-particle Fc-containing proteins using a fluorescence detector, wherein (A) and (B) can be performed consecutively in any order or simultaneously, and (C) comparing the elution time profiles from (A) and (B). The particle can be a virus (for example, adeno-associated virus (AAV)) and the anti-particle Fc-containing protein is an anti-virus antibody (for example, an anti-AAV antibody). Alternatively, the particle is an virus-like particle (VLP) and the anti-particle Fc-containing protein is an anti-VLP antibody, such as an anti-spike protein antibody. The samples can be from the same preparation or different preparations.

The inventions further provide methods for characterizing virus-like particles and viral glycoproteins in a virus-like particle sample using asymmetric flow field-flow fractionation (A4F), wherein the methods comprise the steps of: (A) fractionating by A4F the virus-like particle sample that further comprises a fluorescence labeled detection reagent directed against the viral glycoproteins; and (B) detecting with a fluorescence detector free glycoproteins bound to a fluorescence labeled detection reagent and virus-like particle associated viral glycoproteins bound to a fluorescence labeled detection reagent; and (C) comparing the levels of free glycoproteins and virus-like particle associated viral glycoproteins. The viral glycoproteins can be spike proteins, such as influenza spike proteins, coronavirus spike proteins or Ebola spike proteins. The virus-like particles can be based on one selected from the group consisting of Parvoviridae, Retroviridae, Flaviviridae, Paramyxoviridae, Adenoviridae, vesicular stromatitis virus (VSV) and bacteriophages or other desired virus.

Systems for performing all of the above methods also are provided according to the inventions.

### BRIEF DESCRIPTION OF THE FIGURES

FIG. 1 depicts a schematic representation of an exemplary virus-like particle (VLP). This VLP has a diameter of about 100 nM or greater and a molecular weight of about 100 Megadaltons. Glycoprotein (GP) spikes are on the surface of this exemplary VLP, and thus are VLP-associated GP.

FIG. 2 depicts a graph showing the use A4F-MALS separation to assess the purity of VLPs in six lots.

FIG. 3 depicts A4F-Flr using fluorescently (Flr)-labeled anti-glycoprotein (GP) monoclonal antibodies in order to compare the level of free GP and VLP-associated GP. The left side of FIG. 3 defines the symbols.

FIG. 4 depicts A4F-Flr using fluorescently-labeled anti-glycoprotein monoclonal antibodies in order to compare the level of free GP and VLP-associated GP. Six lots were tested, and the results were consistent. The left side of FIG. 4 defines the symbols.

FIG. 5 depicts data on A4F-MALS separation of AAV-bispecific monoclonal complexes under different ratios of AAV to bispecific antibody (bsAb).

FIG. 6 depicts data on A4F-MALS separation of AAV-monospecific monoclonal antibody (mAb) complexes under different ratios of AAV to mAb.

### DETAILED DESCRIPTION OF THE INVENTIONS

### Definitions

Unless defined otherwise, all technical and scientific terms used herein have the same meaning as commonly understood by one of ordinary skill in the art to which this invention belongs.

The term “about” in the context of numerical values and ranges refers to values or ranges that approximate or are close to the recited values or ranges such that the invention can perform as intended, such as having a desired rate, amount, density, degree, increase, decrease, percentage, value, purity, pH, concentration, presence of a form or variant, temperature or amount of time, as is apparent from the teachings contained herein. For example, “about” can signify values either above or below the stated value in a range of approx. +/−10% or more or less depending on the ability to perform. Thus, this term encompasses values beyond those simply resulting from systematic error.

“Biological fluid(s)” refers to fluids obtained from the body or derived from fluids obtained from the body or contain biological materials, such as proteins. Examples include plasma, serum, cell media, aqueous humor, vitreous humor, interstitial fluid, lymph, synovial fluid, pleural fluid, pericardial fluid, peritoneal fluid and cerebrospinal fluid. This term includes biological fluids that are diluted.

“Fluorescent labels” provide a light signal that can be detected by a fluorescence detector. Exemplary fluorescent labels are well-known in the art, including, but not limited to Discosoma coral (DsRed), green fluorescent protein (GFP), enhanced green fluorescent protein (eGFP), cyano fluorescent protein (CFP), enhanced cyano fluorescent protein (eCFP), yellow fluorescent protein (YFP), enhanced yellow fluorescent protein (eYFP) and far-red fluorescent protein (for example, mKate, mKate2, mPlum, mRaspberry or E2-crimson). See, for example, U.S. Pat. No. 9,816,110. Alexa Fluor dyes that are commercially-available, photostable also can be conjugated to antibodies. These dyes are available with variety of absorption and emission capabilities, and are preferred in instances where there is background biological fluorescence in the biological fluid.

A “fluorescence labeled detection reagent” comprises a fluorescent label (Flr) bound to an Fc-containing protein or a mini-trap protein. An example is an antibody bound to a fluorescent label, and generally referred to as an “Flr-labeled antibody” and other similar terminology.

A “Fractionation buffer” is a buffer a sample is placed and/or diluted in order to conduct fractionation using A4F or SEC. See WO 2020/047067. Buffers are readily attainable and commercially available. Proteins and protein complexes from any source, including biological sources, can be placed in a fractionation buffer.

“Antibodies” (also referred to as “immunoglobulins”) are examples of proteins having multiple polypeptide chains and extensive post-translational modifications. The canonical immunoglobulin protein (for example, IgG) comprises four polypeptide chains—two light chains and two heavy chains. Each light chain is linked to one heavy chain via a cysteine disulfide bond, and the two heavy chains are bound to each other via two cysteine disulfide bonds. Immunoglobulins produced in mammalian systems are also glycosylated at various residues (for example, at asparagine residues) with various polysaccharides, and can differ from species to species, which may affect antigenicity for therapeutic antibodies. Butler and Spearman, “The choice of mammalian cell host and possibilities for glycosylation engineering”, Curr. Opin. Biotech. 30:107-112 (2014).

An antibody includes immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDRI, LCDR2 and LCDR3. The term “high affinity” antibody can refer to those antibodies having a binding affinity to their target of at least 10⁻⁹ M, at least 10⁻¹⁰ M; at least 10⁻¹¹ M; or at least 10⁻¹² M, as measured by surface plasmon resonance, for example, BIACORE™ or solution-affinity ELISA.

The phrase “bispecific antibody” includes an antibody capable of selectively binding two or more epitopes. Bispecific antibodies generally comprise two different heavy chains, with each heavy chain specifically binding a different epitope—either on two different molecules (for example, antigens) or on the same molecule (for example, on the same antigen). If a bispecific antibody is capable of selectively binding two different epitopes (a first epitope and a second epitope), the affinity of the first heavy chain for the first epitope will generally be at least one to two, three or four orders of magnitude lower than the affinity of the first heavy chain for the second epitope, and vice versa. The epitopes recognized by the bispecific antibody can be on the same or a different target (for example, on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (for example, recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with these inventions include those, for example, that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FRI, CDRI, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (i.e., in a wild-type organism) appears between two framework regions in a variable region of a light or a heavy chain of an immunoglobulin molecule (for example, an antibody or a T cell receptor). A CDR can be encoded by, for example, a germline sequence or a rearranged or unrearranged sequence, and, for example, by a naive or a mature B cell or a T cell. In some circumstances (for example, for a CDR3), CDRs can be encoded by two or more sequences (for example, germline sequences) that are not contiguous (for example, in a nucleic acid sequence that has not been rearranged) but are contiguous in a B cell nucleic acid sequence, for example, as the result of splicing or connecting the sequences (for example, V-D-J recombination to form a heavy chain CDR3).

“Antibody derivatives and fragments” include, but are not limited to: antibody fragments (for example, ScFv-Fc, dAB-Fc, half antibodies, Fab), multispecifics (for example, IgG-ScFv, IgG-dab, ScFV-Fc-ScFV, tri-specific). “Fab” refers to an antibody fragment comprising an antigen binding region. An Fab typically will lack the Fc portion.

The phrase “Fc-containing protein” includes antibodies, bispecific antibodies, antibody derivatives containing an Fc, antibody fragments containing an Fc, Fc-fusion proteins, receptor Fc-fusion proteins (including trap proteins), immunoadhesins, and other binding proteins that comprise at least a functional portion of an immunoglobulin CH2 and CH3 region. A “functional portion” refers to a CH2 and CH3 region that can bind a Fc receptor (for example, an FcyR; or an FcRn, (neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional. Fc-fusion proteins include, for example, Fc-fusion (N-terminal), Fc-fusion (C-terminal), mono-Fc-fusion and bispecific Fc-fusion proteins.

“Fc” stands for fragment crystallizable, and is often referred to as a fragment constant. Antibodies contain an Fc region that is made up of two identical protein sequences. IgG has heavy chains known as γ-chains. IgA has heavy chains known as α-chains, IgM has heavy chains known as μ-chains. IgD has heavy chains known as σ-chains. IgE has heavy chains known as ε-chains. In nature, Fc regions are the same in all antibodies of a given class and subclass in the same species. Human IgGs have four subclasses and share about 95% homology amongst the subclasses. In each subclass, the Fc sequences are the same. For example, human IgG1 antibodies will have the same Fc sequences. Likewise, IgG2 antibodies will have the same Fc sequences; IgG3 antibodies will have the same Fc sequences; and IgG4 antibodies will have the same Fc sequences. Alterations in the Fc region create charge variation.

Fc-containing proteins, such as antibodies, can comprise modifications in immunoglobulin domains, including where the modifications affect one or more effector function of the binding protein (for example, modifications that affect FcyR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein (for example, an antibody) and exhibits enhanced serum half-life (as compared with the same Fc-containing protein without the recited modification(s)) and have a modification at position 250 (for example, E or Q); 250 and 428 (for example, L or F); 252 (for example, L/Y/F/W or T), 254 (for example, S or T), and 256 (for example, S/R/Q/E/D or T); or a modification at 428 and/or 433 (for example, L/R/SI/P/Q or K) and/or 434 (for example, H/F or Y); or a modification at 250 and/or 428; or a modification at 307 or 308 (for example, 308F, V308F), and 434. In another example, the modification can comprise a 428L (for example, M428L) and 434S (for example, N434S) modification; a 428L, 2591 (for example, V2591), and a 308F (for example, V308F) modification; a 433K (for example, H433K) and a 434 (for example, 434Y) modification; a 252, 254, and 256 (for example, 252Y, 254T, and 256E) modification; a 2500 and 428L modification (for example, T2500 and M428L); a 307 and/or 308 modification (for example, 308F or 308P).

“Fv” stands for fragment variable, and is primarily responsible for binding to epitopes.

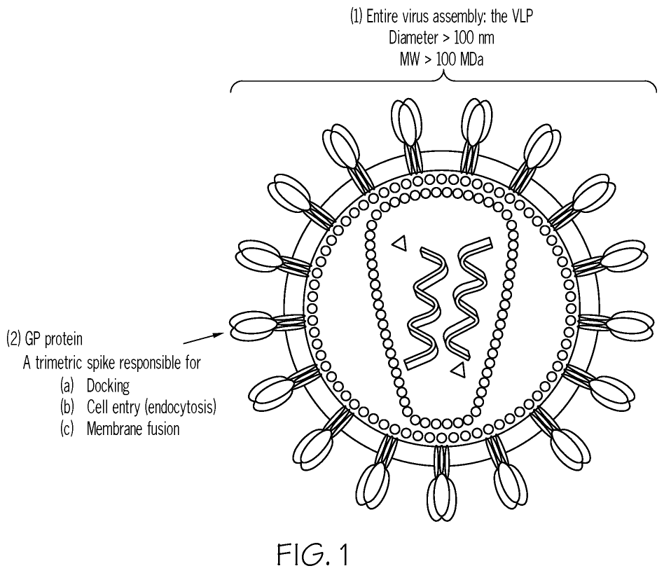

A “virus-like particle” is based on a natural virus, and can be modified to express proteins (for example, glycoproteins (“GP”)) from another virus, such as spike proteins, on the viral capsid. A schematic depiction is shown in FIG. 1.

The term “retargeting molecule” is a molecule useful for directing a virus or virus-like particle, such as a recombinant AAV, to bind to an antigen, receptor and/or ligand found on the surface of a cell. The retargeting molecule can target the cell that has the antigen, receptor and/or ligand that the retargeting molecule can bind to, and thereby direct a recombinant AAV to that cell. Binding can be by way of covalent bonds or non-covalent interactions, such as hydrogen bonds, electrostatic interactions, van der Waals forces, and hydrophobic interactions. For example, a retargeting molecule could be bound to an rAAV capsid by recognition of an epitope by an Fv region of an antibody (for example, a bispecific antibody) or via a specific binding pair, such as the SpyTag-SpyCatcher system. The retargeted rAAV can then target the cell that has the antigen, receptor and/or ligand that the retargeting molecule can bind to. Fc-containing proteins, such as antibodies, monoclonal antibodies (including derivatives, fragments, half antibodies and other heavy chain and/or light chain combinations), multispecific antibodies (for example, bispecifics, IgG-ScFv, IgG-dab, ScFV-Fc-ScFV, trispecifics), Fc-fusion proteins, receptor-Fc fusion proteins, trap proteins can be useful as retargeting molecules. Mini-trap proteins also can be useful as retargeting molecules.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are merely intended to serve as a way of referring individually to each separate value falling within the range, unless otherwise indicated herein, and each separate value is incorporated into the specification as if it were individually recited herein.

### DESCRIPTION

The present inventions combine A4F with Fluorescent (Flr) detectors and optionally MALS detectors to advantageously detect and evaluate viruses and virus-like particles (VLPs). In particular, the present inventions allow for the characterization and evaluation of viruses and VLPs that are bound with proteins from other origins, such as antibodies and proteins from other sources, such as spike proteins from a different type of virus.

### Virus-Like Particles

Virus-like particles, also referred to as pseudoviruses or pseudoparticles, have a number of uses in biologics development. Virus-like particles can be used for assessing the effectiveness of biological therapies.

FIG. 1 depicts a schematic representation of a virus-like particle (VLP). The VLP has a diameter of greater than 100 nM and a molecular weight of over 100 megadaltons. Glycoprotein (GP) spikes are on the surface of this exemplary VLP, and thus are VLP-associated GP. Virus-like particles can be based on a wide variety of virus families including Adenoviridae (such as adenovirus), Parvoviridae (such as adeno-associated virus), Retroviridae (such as lentivirus), Flaviviridae (such as Hepatitis C virus), vesticular stomatitis viruses (VSV), Paramyxoviridae (such as Nipah) and bacteriophages (such as Qβ).

The inner core of the VLP depiction in FIG. 1 shows a nucleocapsid, which is a substructure that includes capsid proteins and the encompassed the genomic nucleic acids. The outer viral capsid is composed of capsid proteins, and in FIG. 1 the viral capsid has viral spike glycoproteins from another type of virus. Spike proteins are responsible for viral docking, cell entry (endocytosis) and membrane fusion. VLPs are useful for determining the activity and effectiveness of an antibody or other type of Fc-containing protein against a pathogenic virus, such as Influenza, Ebola or Covid, for example.

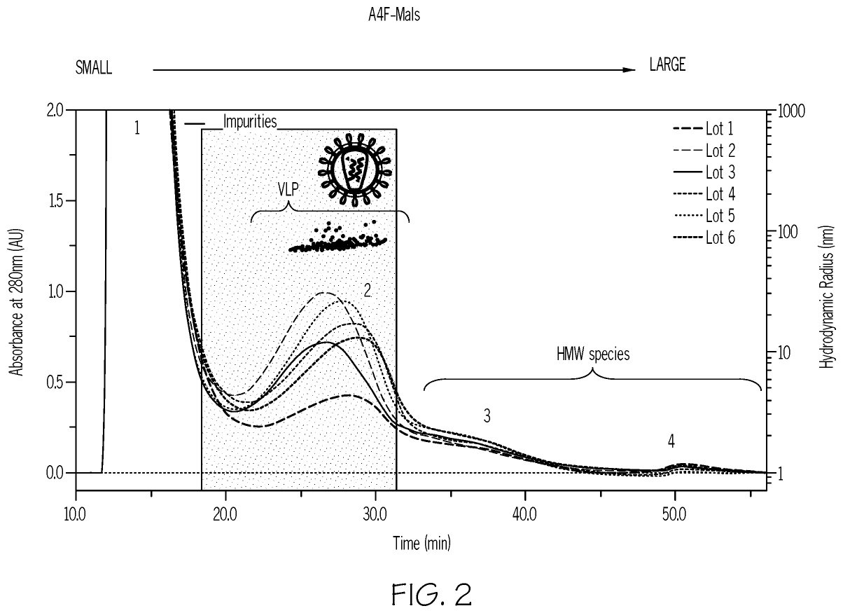

FIG. 2 depicts A4F combined with MALS (A4F-MALS). A4F-MALS was able to separate impurities, VLPs and high molecular weight species in six lots of VLPs. With A4F, lower molecular molecules/complexes elute in less time than higher molecular weight molecules/complexes.

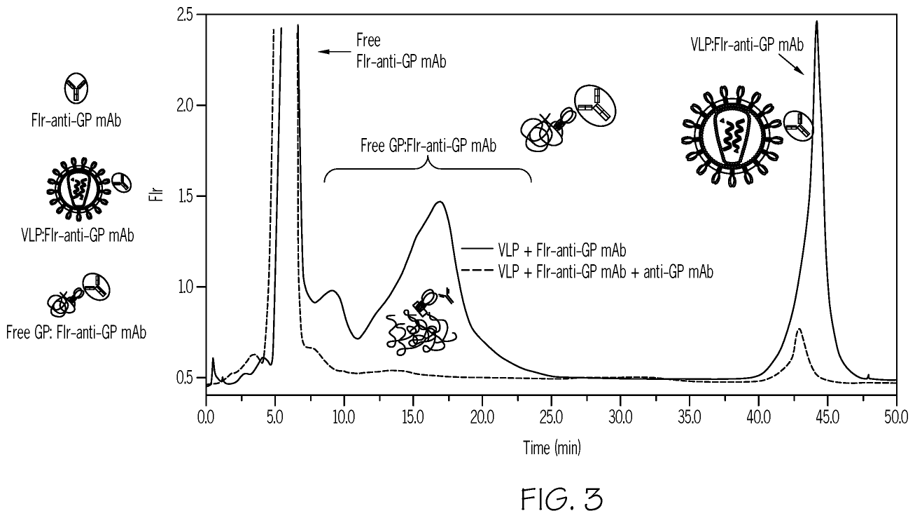

FIG. 3 depicts A4F-Flr using fluorescently (Flr)-labeled anti-glycoprotein (GP) monoclonal antibodies in order to compare the level of free GP and VLP-associated GP. The left side of FIG. 3 defines the symbols.

Two assessments were undertaken as shown in FIG. 3. The first was the VLP with a fluorescently labeled anti-spike glycoprotein monoclonal antibody (Flr-anti-GP-mAb). The second was the VLP with a fluorescently labeled anti-spike glycoprotein monoclonal antibody and an anti-spike glycoprotein monoclonal antibody (anti-GP-mAb), which lacked a fluorescent label. Anti-GP-mAb would be present in an excess amount and compete with Flr-anti-GP-mAb for binding with free GP and the GP bound to the VLP.

FIG. 3 shows that AF4-Flr is able to separate and detect (i) free Flr-labeled antibody, (ii) Flr-labeled antibody bound to free glycoprotein and (iii) Flr-antibody bound to VLP. The diminished signal for detection of (ii) Flr-labeled antibody bound to free glycoprotein and (iii) Flr-antibody bound to VLP when the unlabeled anti-GP-mAb is added to compete with the Flr-antibody establishes that a specific interaction is responsible for fluorescent detection of free GP and the VLP.

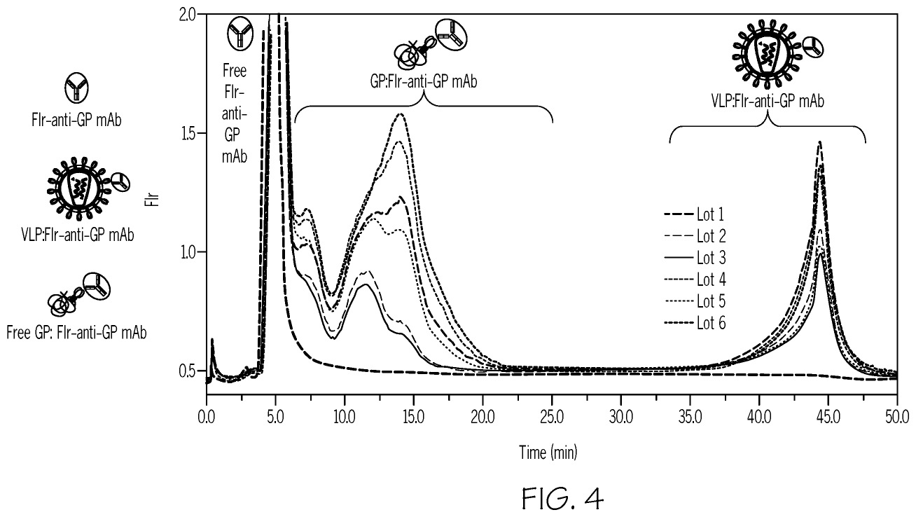

FIG. 4 depicts A4F-Flr using fluorescently-labeled anti-glycoprotein monoclonal antibodies in order to compare the level of free GP and VLP-associated GP. Six lots were tested, and the results were consistent. As expected, (i) free Flr-labeled antibody eluted first, followed by (ii) Flr-labeled antibody bound to free glycoprotein and then (iii) Flr-antibody antibody bound to VLP. The left side of FIG. 4 defines the symbols.

### AAV Retargeting

Adeno-associated virus (AAV) is a non-enveloped, single-stranded DNA virus and is used as a gene delivery vector for both research and therapeutics. Weitzman and Linden, Adeno-Associated Virus Biology (chapter 1), Meth. Molec. Biol. 807:1-23 (2011). There are numerous AAV serotypes and variants thereof. AAV serotypes include, but are not limited to, AAV1, 2, 3, 4, 5, 6, 7, 8, 9, 10, 11, 12, 13, rh74, and others, as well as variants thereof. AAV serotypes share common properties, structure, and genomic sequence and organization. See, for example, Issa et al, Cells 12: 285 (2023); Goedeker et al., Ther. Adv. Neurol. Disord. 16: 1-7 (2023).

Gene transfer vectors based on AAV have demonstrated promise for human gene therapy based on their safety profile and potential to achieve long-term efficacy in animal models. Wang et al., Nature, 18: 358-78 (2019). Recombinant AAVs containing genes of interest (GOIs) are increasingly being used in preventative and therapeutic capacities, such as in vaccines and in gene therapy.

The wild type AAV genome includes a capsid gene referred to as “Cap” or “cap”. Cap in nature is translated to produce, via alternative start codons and transcript splicing, three size-variant structural proteins referred to as VP1 (about 90 kDa), VP2 (about 72 kDa) and VP3 (about 60 kDa). An AAV capsid contains 60 subunits total of the VP proteins. A ratio of 1:1:10 is considered the most typical ratio for VP1:VP2:VP3, with a stoichiometry of 5 VP1 subunits:5 VP2 subunits:50 VP3 subunits. However, there can be variation. Wörner et al., Nature Communications 12:1642 (2021).

Recombinant AAV (rAAV) has been produced in HEK 293, BHK, human amniotic (for example, epithelial cells such as HAEpiC), CHO and Sf9 lines. First generation rAAV was comprised of a GOI replacing the AAV Cap and Rep genes. The GOI would be flanked by AAV inverted terminal repeats (ITRs) so that the GOI could be packaged within an AAV capsid.

Retargeting of recombinant AAVs is undertaken to target the rAAVs to certain cells/tissues and to counteract the tropism that AAV naturally exhibits for the liver. There are a variety of ways to retarget AAV.

One approach for retargeting is to modify the cap gene of AAV to express a short linear epitope. Kuklik et al. employed a nucleotide sequence encoding a “2E3” epitope, which is derived from human proprotein-convertase subtilisin/kexin type 9 (PCSK9). Int. J. Mol. Sci. 22:8355 (2021). The 2E3 nucleotide sequence in the VP proteins could be recognized by antibodies, such as a bispecific antibody (bsAb), that recognize the 2E3 epitope. Kuklik et al. used an anti-2A3 bispecific antibody that also recognized fibroblast activation protein (FAP) or programmed death-ligand 1 (PD-1). The bispecific antibody is bound to the bispecific antibody via the 2E3 epitope, and the rAAV can be retargeted to bid cells expressing FAP or PD-1, as the case may be.

There are other approaches for rAAV retargeting using “specific binding pairs,” also referred to as “protein:protein binding pairs.” An exemplary system is the SpyTag-SpyCatcher system. The SpyTag-SpyCatcher system was developed using the Streptococcus pyogenes second immunoglobulin-like collagen adhesion domain (CnaB2) from the fibronectin binding protein FbaB. An isopeptide bond can be formed spontaneously between the SpyTag protein and the SpyCatcher protein. The SpyCatcher peptide is about 15 kD in size. However, the SpyTag protein is only 13 amino acids long. The small size of the SpyTag protein makes it amenable for insertion into the AAV genome, which has a total packing capacity of only about 4.7 kilobases. A nucleotide sequence encoding the SpyTag peptide can be inserted into the AAV cap gene such the SpyTag peptide can by conjugated to a SpyCathcer protein that is fused to the Fc portion of an antibody. See WO 2019/006046.

Systems to facilitate retargeting include the SpyTag:SpyCatcher system is described in U.S. Pat. No. 9,547,003 and Zakeri et al. (2012) PNAS 109:E690-E697, is derived from the CnaB2 domain of the Streptococcus pyogenes fibronecting-binding protein FbaB. See WO 2019/006046.

SpyTag002:SpyCatcher002 system is described in Keeble et al (2017) Angew. Chem. Int. Ed. Engl. 56:16521-25. See WO 2019/006046.

SpyTag003:Spay Catcher003 also has been created. Spy Tag002:SpyCatcher002 and SpyTag003:SpyCatcher003 are different iterations of Spy Tag:Spy Catcher.

The SnoopTag:SnoopCatcher system is described in Veggiani (2016) PNAS 113: 1202-07. The D4 Ig-like domain of RrgA, an adhesion from Streptococcus pneumoniae, was split to form SnoopTag. Incubation of SnoopTag and SnoopCatcher results in a spontaneous isopeptide bond that is specific between the complementary proteins. Veggiani (2016)), supra. See WO 2019/006046.

The Isopeptag:Pilin-C specific binding pair was derived from the major pilin protein Spy0128 from Streptococcus pyogenes. (Zakeir and Howarth (2010) J. Am. Chem. Soc. 132:4526-27). See WO 2019/006046.

Other systems to facilitate retargeting can be based upon the splitting and engineering of RegA domain 4. These have led to SnoopTagJr:SnoopCatcher, DogTag:DogCatcher and Snoop Ligase. Other systems include Isopeptag:Pilin-N, SdyTg:SdyCatcher, Jo:ln, 3kptTag: 3kptCatcher, 4oq1Taq/4oq1Catcher, NGTag/Catcher, Rumtrunk/Mooncake, GalacTag, Cpe, Ececo, Corio and all others based upon isopeptide binding pairs.

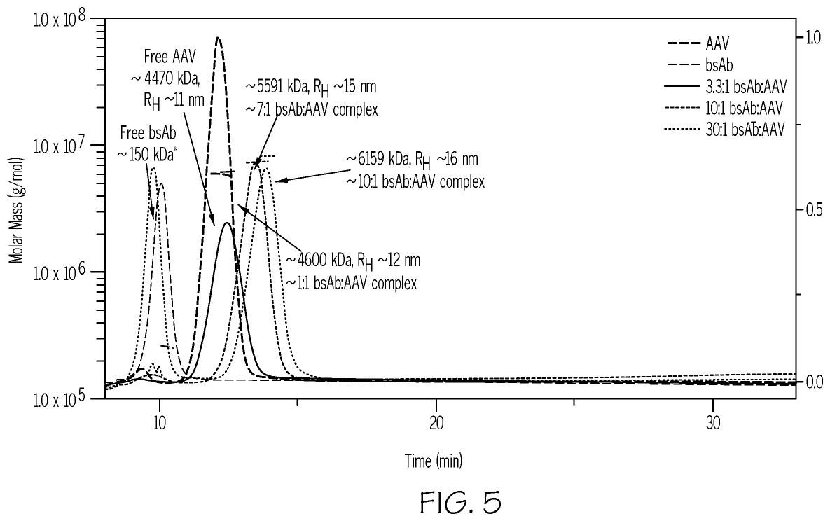

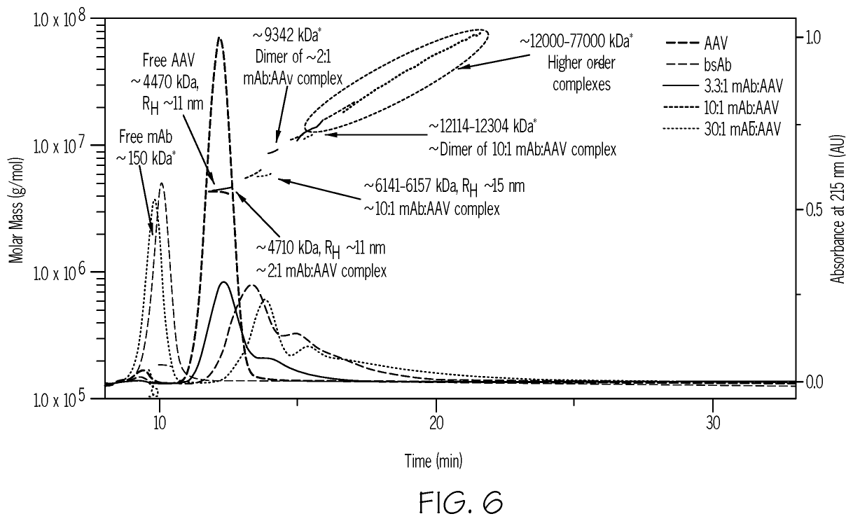

Due to the reactions involved between AAVs and targeting moieties, A4F-MALS and A4F-Flr can be used to elucidate stoichiometry and optimal reaction ratios. For instance, FIG. 5 depicts data on A4F-MALS separation of AAV-bispecific monoclonal complexes under different ratios of AAV to bsAb. FIG. 6 depicts data on A4F-MALS separation of AAV-monospecific monoclonal antibody (mAb) complexes under different ratios of AAV to mAb.

The inventions are further described by the following Examples, which do not limit the inventions in any manner. The order of performance of characterization in the below examples can be altered or combined as determined by the person of skill in the art.

### Example 1—A4F-MALS and A4F-Flr to Characterize Virus-Like Particles

FIG. 2 depicts data from A4F-MALS was able to separate impurities, VLPs and high molecular weight species in six lots of VLPs. Six lots were tested an analyzed, and the results were consistent.

As stated above, with A4F lower molecular molecules/complexes elute in less time than higher molecular weight molecules/complexes. Accordingly, impurities eluted first, and then VLPs eluted between about 20 to 30 minutes, followed by high molecular weight species (HWM). The hydrodynamic radius here was between about 50-60 nm. VLP sizes can vary based upon the type of the VLP.

FIG. 3 depicts A4F-Flr using fluorescently (Flr)-labeled anti-glycoprotein (GP) monoclonal antibodies in order to compare the level of free GP and VLP-associated GP. The left side of FIG. 3 defines the symbols.

Two assessments were undertaken as shown in FIG. 3. The first was the VLP with a fluorescently labeled anti-spike glycoprotein monoclonal antibody (Flr-anti-GP-mAb). The second was the VLP with a fluorescently labeled anti-spike glycoprotein monoclonal antibody and an anti-spike glycoprotein monoclonal antibody (anti-GP-mAb), which lacked a fluorescent label. Anti-GP-mAb would be present in an excess amount and compete with Flr-anti-GP-mAb for binding with free GP and the VLP.

FIG. 3 shows that AF4-Flr is able to separate and detect (i) free Flr-labeled antibody (eluted in about 5-7 minutes), (ii) Flr-labeled antibody bound to free glycoprotein (eluted in about 10-25 minutes) and (iii) Flr-antibody antibody bound to VLP (eluted in about 40-47 minutes). The diminished signal for detection of (ii) Flr-labeled antibody bound to free glycoprotein and (iii) Flr-antibody bound to VLP when the unlabeled anti-GP-mAb is added to compete with the Flr-antibody establishes that a specific interaction is responsible for fluorescent detection of free GP and the VLP.

FIG. 4 depicts A4F-Flr using fluorescently-labeled anti-glycoprotein monoclonal antibodies in order to compare the level of free GP and VLP-associated GP. Six lots were tested, and the results were consistent amongst the lots and the results depicted in FIG. 3. As expected, (i) free Flr-labeled antibody eluted first, followed by (ii) Flr-labeled antibody bound to free glycoprotein and then (iii) Flr-antibody antibody bound to VLP. The left side of FIG. 4 defines the symbols.

### Example 2—A4F-MALS to Characterize Retargeted rAAV

As disclosed above, there are a variety of approaches for retargeting rAAV. FIG. 5 depicts data on A4F-MALS separation of AAV-bispecific monoclonal complexes under different ratios of AAV to bsAb. Table 1 below lists various ratios of AAV bound to bsAb and their theoretical molar masses.

TABLE 1

| Bispecific antibody to AAV ratios | Theoretical Molar Mass (kDa) |
|---|---|
| Free bispecific antibody | 150 |
| Free AAV | 4470 |
| 1:1 | 4620 |
| 3:1 | 4920 |
| 7:1 | 5520 |
| 10:1 | 5970 |
| 20:2 | 11940 |

FIG. 5 depicts data from complexes formed between AAV and bsAbs. The 3.3:1, 10:1 and 30:1 ratios on the right side of the figure are the sample preparation ratios (bsAb:AAV molar ratio). The ratios set forth within the plots are the measured stoichiometry of bsAb-AAV complexes. This data is useful for understanding the stoichiometry and nature of the binding of AAV to the bsAb, and to optimize the AAV to bsAb ratio when producing retargeted AAV.

A4F-Flr can be used to more fully ascertain the nature and efficiency of the reactions. For example, fluorescently (Flr)-labeled bispecific antibody (Flr-bsAb) can be used in a competitive assay with non-labeled bsAb present in an excess amount. A decrease in fluorescent detection of AAV:bsAb complexes would indicate that the complexes are formed by specific binding between the rAAV and the bsAb.

A4F-Flr also can be used to detect and elucidate the stability of AAV:bsAb complexes in biological fluids. For example, AAV:bsAb complexes could be altered in a biological fluid. Depending on the nature of the complex and the biological fluid, complexes could disassociate or otherwise degrade, or complexes could form with varying stoichiometries.

A monospecific antibody also was tested. Table 2 below lists various ratios of AAV bound to monospecific Ab and their theoretical molar masses.

TABLE 2

| Monospecific antibody to AAV ratios | Theoretical Molar Mass (kDa) |
|---|---|
| Free monospecific antibody | 150 |
| Free AAV | 4470 |
| 2:1 | 4770 |
| 3:1 | 4920 |
| 6:1 | 5370 |
| 10:1 | 5970 |
| 20:2 | 11940 |

FIG. 6 depicts data on A4F-MALS separation of AAV-monospecific monoclonal antibody (mAb) complexes under different ratios of AAV to mAb, and is a similar experiment to that of FIG. 5.

The 3.3:1, 10:1 and 30:1 ratios on the right side of the figure are the sample preparation ratios (mAb:AAV molar ratio). The ratios set forth with the plots are the measured stoichiometry of mAb:AAV complexes. This data is useful for understanding the stoichiometry and nature of the binding of AAV to the mAb, and to elucidate the effects of a tested anti-AAV antibody.

A4F-Flr can be used to more fully ascertain the nature and efficiency of the reactions. For example, fluorescently (Flr)-labeled monospecific monoclonal antibody (Flr-mAb) can be used in a competitive assay with non-labeled mAb present in an excess amount. A decrease in fluorescence detection of AAV:mAb complexes would indicate that the complexes are formed by specific binding between the rAAV and the mAb.

A4F-Flr also can be used to detect and elucidate the stability of AAV:mAb complexes in biological fluids. For example, AAV:mAb complexes could be altered in a biological fluid. Depending on the nature of the complex and the biological fluid, complexes could disassociate or otherwise degrade, or complexes could form with varying stoichiometries.

It is to be understood that the description, specific examples and data are given by way of illustration and are not intended to limit the present invention. Various changes and modifications within the present invention, including combining embodiments in whole and in part, will become apparent to the skilled artisan from the discussion, disclosure and data contained herein, and thus are considered part of the invention.

1. A method for characterizing virus-like particles and viral glycoproteins using asymmetric flow field-flow fractionation (A4F) and at least two types of detectors, wherein the method comprises the steps of:
(A) fractionating by A4F a first virus-like particle sample; and determining at least one of molar mass and size distribution of the virus-like particle and viral glycoproteins in the first virus-like particle sample using Multi-Angle Light Scattering (MALS); and
(B) fractionating by A4F a second virus-like particle sample further comprising a fluorescence labeled detection reagent; and detecting the free glycoproteins and virus-like particle associated viral glycoproteins in the second virus-like particle sample using a fluorescence detector,
wherein (A) and (B) can be performed consecutively in any order or simultaneously, and
(C) comparing the elution time profiles from (A) and (B).

2. The method according to claim 1, wherein the viral glycoproteins are spike proteins.

3. The method according to claim 2, wherein the spike proteins are influenza spike proteins.

4. The method according to claim 2, wherein the spike proteins are a coronavirus spike proteins.

5. The method according to claim 3, wherein the spike proteins are Ebola spike proteins.

6. The method according to claim 1, wherein the virus-like particles are based on a retrovirus, an adenovirus, a vesicular stomatitis virus (VSV), parvovirus, flavivirus, paramyxovirus, or bacteriophage.

7.-12. (canceled)

13. A method for characterizing retargeted adeno-associated virions (retargeted AAV) and retargeting molecules using asymmetric flow field-flow fractionation (A4F) and at least two types of detectors, wherein the method comprises the steps of:
(A) fractionating by A4F a first retargeted AAV sample; and determining at least one of molar mass and size distribution of retargeted adeno-associated virions and retargeting molecules in the first retargeted AAV sample using Multi-Angle Light Scattering (MALS); and
(B) fractionating by A4F a second retargeted AAV sample further comprising a fluorescence labeled detection reagent; and detecting the retargeted adeno-associated virions and retargeting molecules in the second retargeted AAV sample using a fluorescence detector,
wherein (A) and (B) can be performed consecutively in any order or simultaneously, and
(C) comparing the elution time profiles from (A) and (B).

14. The method according to claim 13, wherein the retargeting molecule is a bispecific antibody.

15. The method method of claim 13, wherein the AAV comprises a transgene.

16. The method of claim 13, wherein the AAV is selected from the group of AAV1, AAV2, AAV3, AAV4, AAV5, AAV6, AAV7, AAV8, AAV9, AAV10, AAV11, AAV12, AAV13 and AAVrh74.

17. A method for characterizing samples comprising (i) particles selected from the group consisting of viruses and virus-like particles and (ii) anti-particle Fc-containing proteins, wherein the characterizing is by using asymmetric flow field-flow fractionation (A4F) and at least two types of detectors, wherein the method comprises the steps of:
(A) fractionating by A4F a first particle sample; and determining at least one of molar mass and size distribution of particles and Fc-containing proteins using Multi-Angle Light Scattering (MALS); and
(B) fractionating by A4F a second particle sample further comprising a fluorescence labeled detection reagent; and detecting the particles and anti-particle Fc-containing proteins using a fluorescence detector,
wherein (A) and (B) can be performed consecutively in any order or simultaneously, and
(C) comparing the elution time profiles from (A) and (B).

18. The method according to claim 17, wherein the particles are adeno-associated virus (AAV) and the anti-particle Fc-containing protein is an anti-AAV antibody.

19. The method according to claim 17, wherein the particles are virus-like particles (VLP) and the anti-particle Fc-containing protein is an anti-VLP antibody.

20. The method according to claim 19, wherein the anti-VLP antibody is an anti-spike protein antibody.

21. A method for characterizing virus-like particles and viral glycoproteins in a virus-like particle sample using asymmetric flow field-flow fractionation (A4F), wherein the method comprises the steps of:
(A) fractionating by A4F a virus-like particle sample further comprising a fluorescence labeled detection reagent directed against the viral glycoproteins; and
(B) detecting with a fluorescence detector free glycoproteins bound to a fluorescence labeled detection reagent and virus-like particle associated viral glycoproteins bound to a fluorescence labeled detection reagent; and
(C) comparing the levels of free glycoproteins and virus-like particle associated viral glycoproteins.

22. The method according to claim 21, wherein the viral glycoproteins are spike proteins.

23. The method according to claim 22, wherein the spike proteins are influenza spike proteins, coronavirus spike proteins, or Ebola spike proteins.

24.-25. (canceled)

26. The method of claim 21, wherein the virus-like particles is based on one selected from the group consisting of Parvoviridae, Retroviridae, Flaviviridae, Paramyxoviridae, Adenoviridae, vesicular stomatitis virus (VSV) and bacteriophages.

27. A system for performing the method of claim 1.

28. A system for performing the method of claim 13.

29. A system for performing the method of claim 17.

30. A system for performing the method of claim 21.
